(19) United States
(12) Patent Application Publication
(10) Pub. No.: US 2004/0087832 A1
(43) Pub. Date: May 6, 2004

(54) Device and method for blocking activation of an in-vivo sensor

(76) Inventors: Arkady Glukhovsky, Santa Clarita, CA (US); Dov Avni, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/695,847

(22) Filed: Oct. 30, 2003

Related U.S. Application Data
(60) Provisional application No. 60/422,113, filed on Oct. 30, 2002.

(51) Int. Cl. A61B 1/00

(52) U.S. Cl. 600/118

(57) ABSTRACT

There is provided an in-vivo device, for example an imaging device, configured with operation blocking capability to prevent the reuse of such device or to prevent a reactivation of such device after such device has completed a medical examination.

### CROSS REFERENCE TO RELATED APPLICATIONS

The present application claims benefit from prior provisional patent application serial No. 60/422,113 filed on Oct. 30, 2002 and entitled “DEVICE AND METHOD FOR CONTROLLING THE OPERATION OF AN IN-VIVO SENSOR”, incorporated herein by reference in its entirety.

### FIELD OF THE INVENTION

The present invention relates to a method for controlling the operation of an in-vivo sensor, for example, an imaging sensor within an ingestible device.

### BACKGROUND OF THE INVENTION

Ingestible electronic devices such as devices which are moved through the digestive tract through the action of, for example, peristalsis and which collect data and transmit the data to a receiver system are known. These devices may be utilized to measure, for example, pH, temperature and pressure throughout the intestines. The devices may contain electrical components such as an imaging system for obtaining images from inside a body lumen, which may be transmitted to an external receiving unit.

The electrical components within the device are generally powered by a battery which generally has a limited life span, sufficient for its operational use. It should be noted that alternative energy sources may be utilized, e.g. wireless energy delivery to the device from an external charging device. It may be possible to reuse the components of the device by retrieving it upon excretion and replacing the battery (if needed). Re-using an in-vivo device, which has been designed or intended for a single use may have drawbacks.

### SUMMARY OF THE INVENTION

An embodiment of the present invention relates to an ingestible device such as for example a capsule (other suitable configurations, shapes and containers may be used) which may contain an in-vivo sensor, such as an imaging sensor, and which may for example transmit signals to an external receiving unit and which may be configured to be used for a single use such that it is non-reusable. According to an embodiment of the present invention, removal or replacement of the battery does not suffice to reactivate electrical or other components of the device or to deactivate an operation blocker.

An embodiment of the present invention may permanently prevent or block the device from being reused or reactivated once a specified condition is satisfied. An embodiment of the invention may restrict the activation of operational capabilities of, for example, a sensor or imaging system contained within the device and/or prevent activation of a transmitter or other component within the device from transmitting signals, for example, signals from the imaging system.

According to a further embodiment of the present invention, a device may be operated (switched on and off), for example, during manufacturing tests, while an embodiment of the invention may block the device from being used for more than a single typical medical examination or other regular use. Such blockage is, in some embodiments, permanent, provided that abnormal tampering or reconstruction of the device does not take place.

### BRIEF DESCRIPTION OF THE DRAWINGS

The present invention will be understood and appreciated more fully from the following detailed description taken in conjunction with the drawings in which:

FIG. 1 is a schematic illustration of an exemplary in-vivo sensor contained within an ingestible device;

FIG. 2 is a block diagram depicting a device according to an embodiment of the invention;

FIG. 3 is a timing diagram for the operation of a device according to an embodiment of the invention;

FIG. 4 is a schematic block diagram of a typical transmitter for use with an in-vivo imaging system, according to an embodiment of the invention;

FIG. 5 is a schematic flow chart diagram presentation of a method according to an embodiment of the invention;

FIG. 6 is a schematic flow chart diagram presentation of a method according to another embodiment of the invention; and

FIG. 7 is a schematic flow chart diagram presentation of a method according to certain embodiments of the invention.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Embodiments of the present invention relate to an ingestible device such as a capsule which may contain for example an in-vivo sensor, such as an imaging device, which has been configured to be used for a single operation or use.

It will be appreciated by persons knowledgeable in the art that though reference is herein made to an ingestible capsule containing an in-vivo sensor such as, for example, an imaging device, the present invention is also applicable to in-vivo devices, other than capsules and/or that do not have imaging capabilities. For example, an in-vivo device may contain other types of sensing devices, such as pH, temperature, pressure, electrical, impedance and biosensors. All of these sensors are known in the art and will not be described in further detail. Furthermore, more than one sensor may be incorporated within an in-vivo device, allowing for multiple sensing functions to be carried out.

Embodiments of the device are typically autonomous and are typically self contained. For example, the device may be an in-vivo device where all the operative components are substantially contained within a container, and where the device does not require any wires or cables to, for example, receive power or transmit information. For example, power may be provided by an internal battery or a wireless receiving system. Other embodiments may have other configurations and capabilities. For example, components may be distributed over multiple sites or units. Control information may be received from an external source.

Examples of in-vivo sensors that may be used in the present invention are described in U.S. Pat. No. 5,604,531 to Iddan entitled “An In-vivo Camera Video System”, and in International Application Publication No. WO 01/65995, entitled “A Device and System for In-Vivo Imaging”, both of which are assigned to the common assignee of the present invention and are hereby incorporated herein by reference. Other suitable sensing devices may be used. Embodiments of the present invention may use a receiving, processing, and/or display system as disclosed within WO 01/65995 and/or in U.S. Pat. No. 5,604,531 to Iddan; other suitable receiving, processing, and/or display systems may be used.

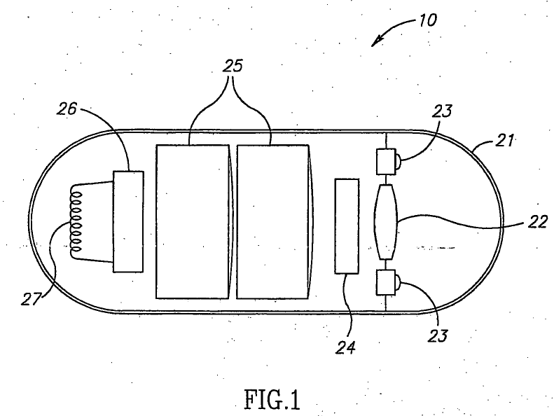

Reference is now made to FIG. 1, which is a schematic illustration of an exemplary in-vivo sensor contained within a device such as, for example, an ingestible device.

The device 10 typically comprises an optical window 21 and an imaging system for obtaining images from inside a body lumen. Device 10 may be a self-contained device, such as an ingestible device, but may have other configurations; for example, the device 10 need not be a capsule. The imaging system may include one or more illumination source(s) 23, such as a white Light Emitting Diode (LED), a Complementary Metal Oxide Semiconductor (CMOS) or other suitable imager 24, which may detect images and an optical system 22 which may focus images onto, for example, the imager 24; other suitable imaging devices, such as CCD devices, may be used. Other suitable illumination sources may be used. Illumination source 23 may illuminate inner portions of the body lumen through optical window 21. Device 10 may further include tansmitter/controller 26 and an antenna 27 or other components for transmitting, for example, image signals of the imager 24 or other signals, and a power source 25, such as for example silver oxide batteries, that may provide power to the electrical elements of the device 10. The transmitter/controller 26 may include, for example, an Application Specific Integrated Circuit (ASIC); other suitable components may be used. While in one embodiment control and transmission capabilities are in the same unit, in other embodiments such capabilities may be located in different components of device 10. Device 10 may include a control unit 500 that may be or include for example circuits, memory, and operation blockers in accordance with an embodiment of the invention. As described below, in some embodiments, components of unit 500 may be configured in a single unit or location. In some embodiments, unit 500 may be included in transmitter/controller 26. In some embodiments, such components of unit 500 may be located in one or more other locations in device 10. Other or additional components may be included in unit 500.

A device such as an ingestible device may be kept inactive, for example, while contained in a package having a magnet, such as the magnetic packaging described in PCT application IL00/00752, filed on Nov. 25, 2000, entitled “Method for Activating an Image Collection Process”, which is assigned to the common assignee of the present invention and which is hereby incorporated in its entirety by reference. Just prior to use or at another time, the wrapping or packaging containing the magnet may be removed causing a switch to be opened, thereby activating the controller, which activates or enables operation of the other system modules, such as for example the transmitter, imager and illumination or other components or systems of the device 10. It should be appreciated that activation and deactivation of the device 10 (e.g., switching it on and off, or switching certain components on and off, or changing from an active to an inactive mode) may be effected by methods other than magnetic, e.g. by mechanical pressure, moisture or other environmental conditions or signal from an external source.

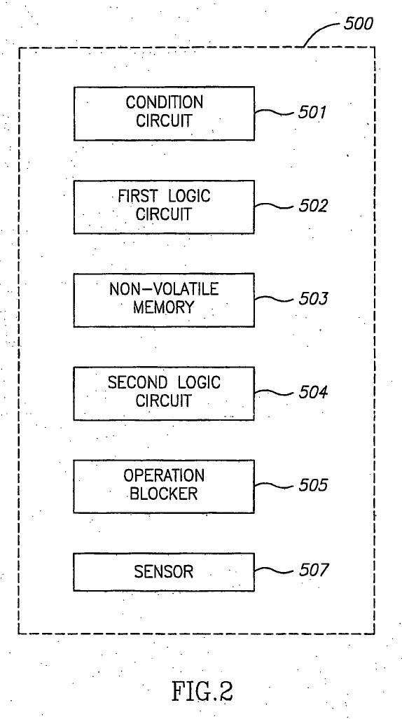

Reference is now made to FIG. 2 in which a block diagram of components of a device according to one embodiment of the invention is illustrated. According to embodiments of the invention a unit 500 of device 10 may include condition circuit 501, which may relay one or more signals to a first logic circuit 502. The first logic circuit 502 may determine whether the conditions (for example, as relayed by the condition circuit 501) are appropriate for changing the logical state of a memory, for example by, burning a memory such as for example a non-volatile memory 503 into an ON state, where such memory is capable of assuming and retaining a designated state. A second logic circuit 504 may determine whether the non-volatile memory 503 has been burned, and may activate an operation blocker 505 accordingly. According to one embodiment the condition circuit 501 may include a comparator for determining whether a certain threshold (for example, a voltage level in a battery falling below a voltage threshold, as further detailed below) was reached or exceeded. According to further embodiments a comparator may determine whether a certain in-vivo parameter (such as pH, temperature, pressure etc.) has been reached. According to yet other embodiments the comparator may include a counter or timer that may determine whether a predetermined time period has lapsed, or whether a predetermined number of image frames or other sensory readings have been obtained by an in-vivo imager or other sensor, etc. According to another embodiment the condition circuit 501 may include a circuit which changes its output according to a command from an external transmitter that may be received by device 10. In some embodiments, some or all of the components in unit 500 may be configured in transmitter/controller 26. In some embodiments, components of unit 500 may be configured inside or external to device 10.

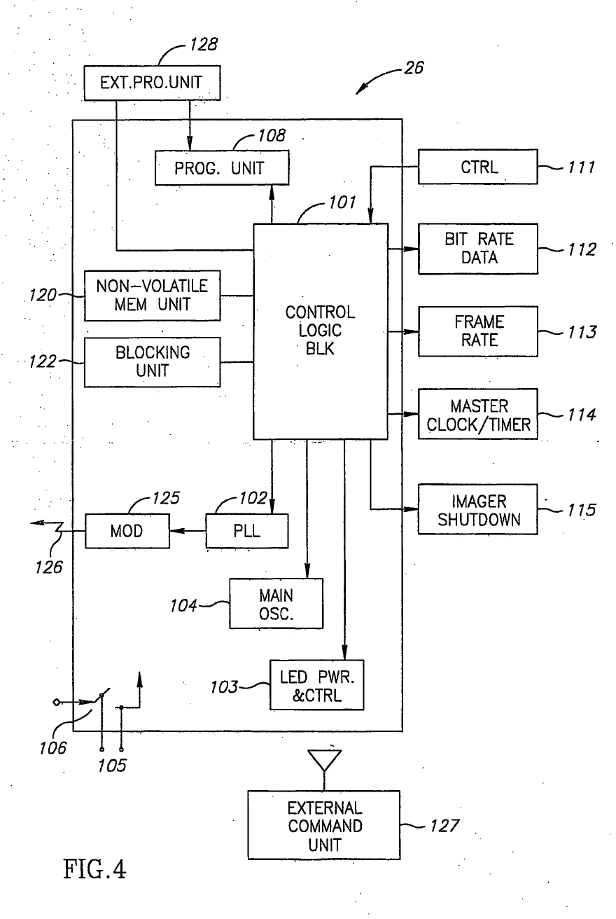

Once a certain condition (or conditions) is fulfilled, as determined for example by first logic circuit 502, a memory such as a non-volatile memory 503 may be burned to serve as a signal for eventual or permanent irreversible prevention of reactivation of an in-vivo sensing device, in which a device according to embodiments of the invention is implemented. A device according to embodiments of the invention may be implemented in internal components of an in-vivo sensing device, such as for example a device described above. Alternatively, the device according to embodiments of the invention may be implemented in components external to an in-vivo sensor, such as in an external command unit located outside of a body as is described in FIG. 4 below. Device 500 may also include one or more sensors 507 such as for example a pH sensor, pressure sensor or other sensors of an environmental condition in a body lumen.

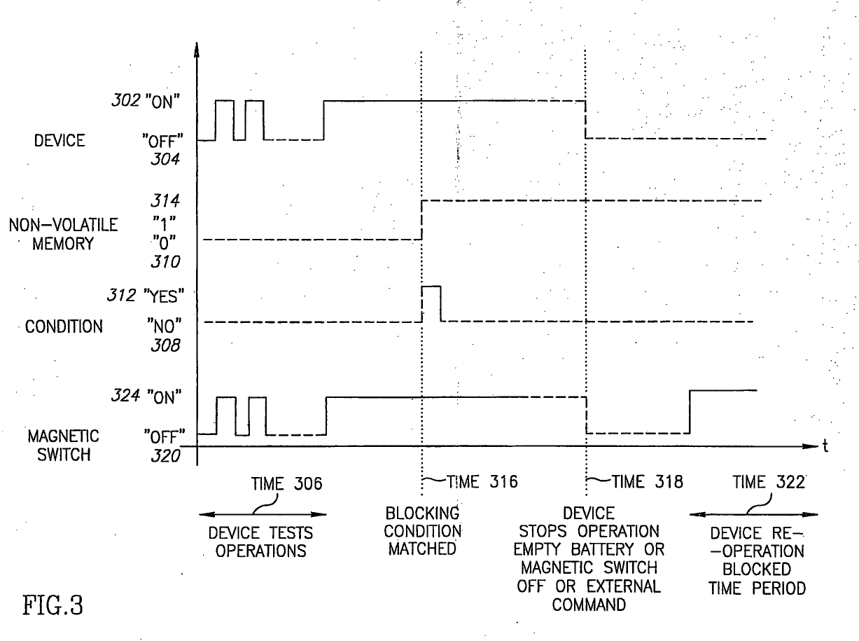

A timing diagram for a device operating in accordance with an embodiment of the invention is presented in FIG. 3. In some embodiments, a device or components within a device such as an ingestible device 10, may be activated or deactivated to an “on” 302 or “off” 304 state in synchronization or as controlled by, for example, a magnetic switch, at least until blocking conditions are matched, as described below. Other manners of initiating operation may be used, and a magnetic switch may not be required. During a period, for example a period of device 10 testing (306) and until blocking conditions are matched, the device may be turned “on” 302 and “off” 304 several times, for example, while testing the device's 10 operation or for others reasons. A testing period need not be used or accounted for. As long as a predefined or blocking condition is not met or satisfied (e.g., condition is “no” 308), a non-volatile memory 503 of device 10 is not burned (non-volatile memory is “0” 310) or another activation blocker is not turned on. However, once blocking conditions are matched in time 316 (e.g., condition goes to “yes” 312) the non-volatile memory 503 may be burned so that non-volatile memory is “1” 314, or some other activation blocker is turned on or activated. The burning may be irreversible (e.g., non-volatile memory 503 becomes perpetually “1” 314). In other embodiments, other manners of recording that operation of device 10 should not resume may be used. For example, a memory bit or register may be set, a fuse or connection may be altered, a component (e.g., an imager) may be physically or logically disabled or damaged. In some embodiments a device's 10 operation may remain “on” and may continue until an appropriate event causes the device's 10 operation to be “off” in time 318. An appropriate event may include, for example, a magnetic switch being turned “off” 320, the device's 10 battery being depleted or reaching a certain low power level, an external command being received, and so on. Other causes for the deactivation of a device 10 are possible.

In some embodiments, once the device's 10 operation is “off” 304 or deactivated in time 318, and the non-volatile memory 503 is burned to “1” 314 or the other operation blocker is turned on (see the time period 322 identified as “Device re-operation blocked”), device's 10 operation or one or more functions operating within a device 10 will not be able to be activated or turned “on” 302 again or reused, regardless of the then current conditions of the device 10 or the then satisfaction of the specified conditions and regardless of the magnetic switch (even if the magnetic switch is turned “on” 324).

In some embodiments, matching of blocking conditions or satisfaction of predefined conditions for activating an operation blocker 505 may coincide with the completion or approximate completion of a single use (or an expected single use) of the device in its regular or typical usage. For example, an in-vivo device 10 or a circuit or component of such in-vivo device 10 that, for example, captures images or other sensory data of a body lumen, may be configured to activate an operation blocker 505 once certain specified conditions have been satisfied. Such conditions may for example reflect the approximate time period that typically elapses in such a single or typical use, an environmental condition that may generally be encountered or sensed by such device 10 in the course of a single or typical use, or the completion by such device 10 of one or more tasks or series of tasks that would generally be completed in a single or typical use. Such conditions may not coincide with a brief or atypical use or operation of the device 10, such as for example a use or condition that may occur in a testing or aborted use of an in-vivo device 10.

A typical use of a device 10 (e.g., an in vivo imaging capsule) may be measured, for example, in hours, whereas a typical use may be in the range of a few minutes (e.g., for imaging a specific part of the GI tract, such as the esophagus) to a few hours (e.g., for imaging the entire GI tract). According to another embodiment a typical use of a device 10 may be determined by environmental pH (e.g., low pH ranges for sensing the stomach). Other parameters may be used for typical uses according to embodiments of the invention.

In an exemplary embodiment of the invention, the current operation of an in-vivo device 10 that had already been activated may not be interrupted or otherwise impaired when a blocking condition is satisfied or an operation blocker 505 is activated. Rather, such blocking condition may prevent future activations of device 10; thus the blocking operation may be permanent. In some embodiments a satisfaction of a predefined condition may cease the activation of one or more functions of a device 10, and block or prevent further activations, or turning “on”, of such device 10.

Reference is now made to FIG. 4 in which a schematic block diagram of a transmitter/controller 26 according to an embodiment of the invention is illustrated. The transmitter/controller 26 of FIG. 4 may be used with an in-vivo imaging system, for example, the imaging system of FIG. 1. The transmitter/controller 26 may be or include an ASIC (application specific integrated circuit), which may for example operate on a minimum shift keying (MSK) modulation system to effect transmitting of digital signals through antenna 126 to a receiving system. Other suitable components for providing suitable functionality may be used. The transmitter/controller 26 may include for example a one time programming unit 108 in communication with external programming input 128 (used, for example, during device 10 manufacturing process), a control logic block 101 for communicating with the imager, a phase lock loop (PLL) 102 in communication with modulator 125, optionally, a LED power and control block 103 for controlling the illumination, a main oscillator 104 and a magnetic switch 105 which may for example control an internal electronic switch 106. In some embodiments, an external command unit 127 may broadcast signals from, for example, outside transmitter/controller 26 or outside of a device or a body, ordering a transmitter controller 26 or some other component of a device 10 to burn a non-volatile memory unit 120 or otherwise activate an operational blocking unit 122. In some embodiments, an external command unit 127 (such as a receiving, processing or display system as disclosed in embodiments of the invention described in U.S. Pat. No. 5,604,531 to Iddan entitled “An In-vivo Camera Video System”, and in embodiments of the invention described in U.S. patent application Ser. No. 09/800,470 to Iddan et. al., entitled “A Device and System for In-Vivo Imaging” published on Nov. 1, 2001 as Pub.# U.S. 2001-0035902 A1 may be operably connected to a receiving unit that may receive signals from an in-vivo device 10. External command unit 127 may transmit instructions by for example radio signals that may be received by a receiver within device 10. Other configurations of transmitter/controller 26 are possible, where less than all of such components are included. Other components and functionalities may also be included.

According to an embodiment the control logic block 101 may for example communicate with components of the in-vivo device (e.g., the imager), read preprogrammed parameters and perform as an interface to the “outside” world in the programming mode. Control logic block 101 may for example maintain a master clock (which may contain, for example, a counter, a timer, etc.). Control logic block 101 may be synchronized by, for example, bit rate data 112, by frame rate 113, or through control 111, which may trigger LED power and control block 103. Control logic block 101 may further control the master clock/timer 114 and the imager shutdown 115.

According to an embodiment of the present invention, the transmitter/controller 26 may be programmed for example to begin transmitting signals after a pre-determined delay. For example, it may take 2-8 hours for the device 10 to reach the large intestine and during this time, the transmitter/controller 26 may be set in shutdown mode, for example, to conserve the power supply of the device 10. During shutdown, the imager and other device electronics may be switched off and the transmitter/controller 26 may send out beacon signals only, or no signals.

According to an embodiment, the transmitter/controller 26 may be controlled by an external magnetic switch 105. The switch 105 may be for example a normally opened (NO) switch that may be kept closed by an external magnet, for example, a magnet in the device wrapping. Other methods of controlling the magnet switch 105 may be used, for example, by applying mechanical pressure. The NO switch may be controlled by an external magnet that keeps the switch closed while it is in proximity to the switch. However, an internal block maintains the logistics of an open switch, so as to keep the transmitter/controller 26 circuits and all device main subsystems inactive while the external magnet is present. Removal of the external magnet causes the switch to open and the internal block to close, thereby allowing the transmitter circuits and device main subsystems to be activated. It should be appreciated that a Normally Closed (NC) magnetic switch (e.g., a Reed Switch) may be used. Additional switch embodiments may be considered, such as a switch operated by application of an external mechanical force through a flexible area of the device 10 envelope. Other methods of controlling or initiating operation of the in-vivo device 10 may be used.

Switch 105 may control for example an internal electronic switch 106 that may control some or all of the device 10 electronics and components. According to one embodiment, electronic switch 106 may include, for example, a low leakage circuitry to convert the logic of the NO switch 105 to NC logic, such that although switch 105 is a NO switch it will keep the transmitter/controller 26 inactive while it is closed.

In an embodiment of the present invention, the ASIC of the transmitter/controller 26 or another component may include an operational blocking unit 122. The operational blocking unit 122 may be set to “On” or “Off” for determining the operational status of the device 10 or the operational status of different modes of the device 10, e.g., imaging, image transmitting, illumination etc. According to an embodiment the operational blocking unit 122 may be a circuit that may be activated or set to “On”, under certain conditions (such as conditions discussed herein), In order to disable some or all of device's 10 operations, so that re-use of the device 10 is avoided or prevented. In some embodiments, operation blocking unit 122 may be a circuit such as for example a circuit included in a non-volatile memory. In other embodiments, operation blocking unit 122, may be a physical device or an absence of a physical device, such as for example an insulator that may be melted by for example a charge, on the satisfaction of certain conditions. Operation blocking unit 122 may be, for example, a memory bit or register that may be set to prevent future operation. Operation blocking unit 122 may be a component such as an imager or logic control block, that is physically or logically disabled to prevent future use, such as a fuse, a physical switch, etc.

In an embodiment of the present invention, the transmitter/controller 26 (e.g., ASIC or other suitable component(s)) may further include a non-volatile memory unit 120. According to an embodiment, the memory unit 120 may record the operational time of the system or the time during which the device has been operational. The control logic block 101 may check the memory unit 120 and if a pre-determined operational time of the memory unit 120 has been exceeded, logic block 101 or some other component may set the operational blocking unit 122 to “On”. The device 10 may continue to transmit until the battery is exhausted. A predetermined time (for example, 3 hours of operation) may be a time exceeding the time typically spent on testing a device 10 for errors. For example, during manufacture, the electrical components of the device 10, including for example the imaging system, transmitter and data line may be tested. Testing may include, for example, switching the entire device 10 or its modules on and off several times, for example, to test the image and transmission quality. In other cases, a device 10 may be activated but not used (e.g., a patient may decide he does not want to ingest the device 10 after the magnetic packaging of the device has been removed, thereby aborting the medical exam). Thus, it may not be sufficient for the control logic block 101 to determine only whether the system has been previously operated in order to activate an operation blocker 122 or switch off or block further operations. In some embodiments, a predetermined time may be a period that is less than the time that it generally takes for the device 10 to pass through a body or otherwise perform a medical examination.

In alternative embodiments the operational blocking unit 122 may be set to indicate non-operation once the battery voltage has reached a predetermined low level, e.g. under 2.7-2 volt (other suitable values may be used), indicating that the battery operation is about to end. In some embodiments, once the magnetic switch 105 has been operated, the control logic block 101 determines whether the operational blocking unit 122 is set to “On” and if affirmative does not allow the device 10 to reactivate or become operational. Thus, according to embodiments of the invention, even if a battery is replaced, a device 10, which has been previously used or exceeded its operational time will not be able to restart or reactivate its operation.

It will be appreciated by persons knowledgeable in the art that the non-volatile memory and operation logic circuits are not restricted to the ASIC of the transmitter/controller 26 but may be implemented in other internal components such as the imager 24, illumination devices 23 or in external components.

As is known in the art, there are various types of suitable non-volatile memory that may be used or included in operational blocking units 122. For example, a non-volatile memory cell may include a PN junction (diode), which may be “burned” with certain voltage. In this case, the non-volatile memory cell may include a diode and a circuit for generating voltage for burning the non-volatile memory. According to one embodiment of the invention, the cell may be implemented on a silicone die (e.g., a chip), which may then be added to a suitable component. It should be appreciated that other types of non-volatile memory may be considered, including mechanical micro-mechanical devices which change status by application of electrical voltage/current.

The status of the non-volatile memory (that is, the burning of the circuit into an activated position to facilitate operation blocking) may be altered by a suitable pre-determined criteria or conditions. Non-limiting examples of criteria may include:

the total elapsed operation time that the device 10 (as measured for example by a counter which may measure the elapsed time by for example counting the master clock pulses or some other time keeping device), continues its operation and is not reset each time that the device 10 is shut down and switched on again);

the time elapsed since the current operation began (that is, the counter is reset each time);

the number of images that may have been captured by the device 10;

the number of times that the device was switched on;

upon reaching a pre-determined voltage level, for example when the battery reaches a specific depletion level;

according to pre-determined physical or chemical parameters (for example, endo-luminal pH, temperature, and/or pressure) that may have been detected in an environment external to device 10;

upon receiving an external command. In this embodiment, the device 10 may include a receiver and decoder in order to receive and interpret the external command. Other external signals are possible.

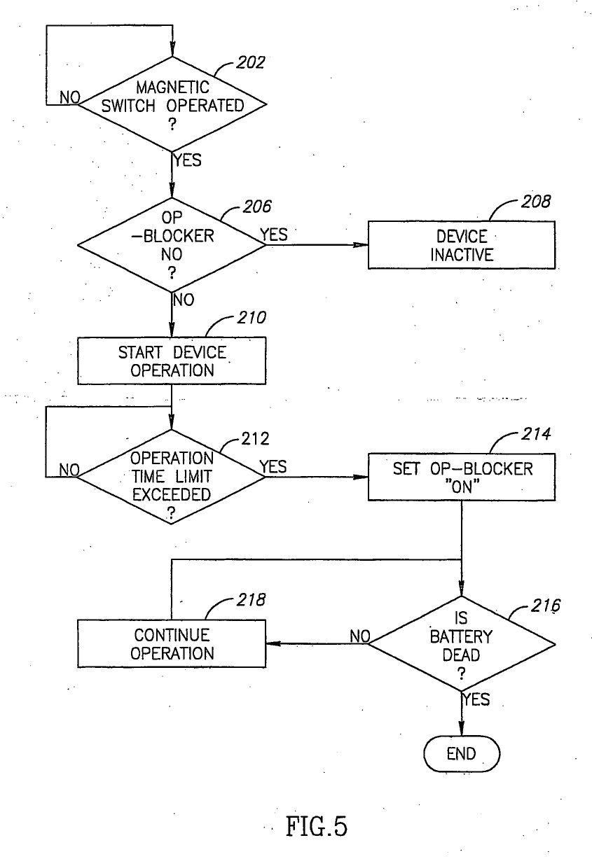

Reference is now made to FIG. 5, in which a schematic flow chart diagram of a method, according to an embodiment of the invention, is illustrated. In some embodiments, the transmitter/controller 26 or another component may check whether the magnetic switch has been activated (block 202). If the magnetic switch has not been activated, such checking may continue. If the magnetic switch 105 has been activated, a check is made to determine whether the operation blocker has been activated or set to “ON” (block 206). If the “Op-blocker” is set to “N”, the device 10 remains inactive (block 208). Inactivation may include blocking all device operations or just certain modes, such as imaging or image transmitting. In some embodiments, if the operational blocking device is, for example, a component that is disabled (e.g., an imager) or a component that disables other components (e.g., a fuse), its status need not be “read” or determined by other components. In such cases, the overall in-vivo device 10 does not operate properly or at all without the operation of the required component (e.g., no images are captured). In other embodiments the blocking may not be permanent. “If the “Op-blocker” is set to “OFF”, the device operation may be activated (block 210). A continuing or periodic check may in some embodiments be made to determine whether the operation time for the device 10 has been exceeded (block 212). If the device is still operating within its specified time limit, the device 10 operation continues. The specified time limit may be any time period larger than the time typically taken to test a device 10 during production or the time it might take a doctor to discover that a device 10 has been activated in error. Other time limits may be used. Typically, the time limit indicates continuous, or normal use of a device 10 in a current operation session (usually in the range of hours as compared to minutes in the case of mistakes). Once the specified operational time for a session of operation of the device has been exhausted, the “Op-blocker” may be set to “ON” (block 214). In some embodiments, operation of the device 10 may continue but reactivation of the device 10 once it ceases operation may be prevented. In some embodiments, the device 10 may be permitted to operate (block 218) until for example its battery dies (block 216) and it runs out of operational power and ends operation.

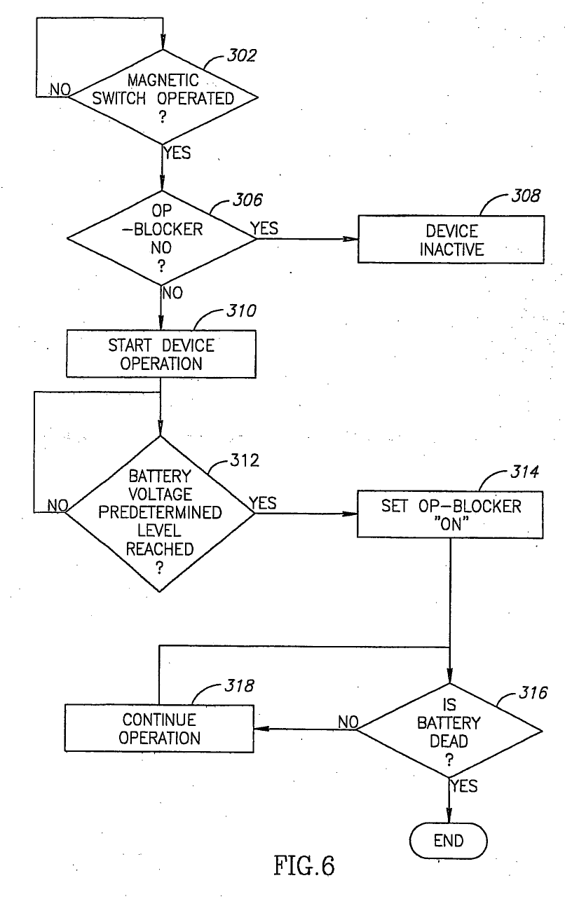

In an alternative embodiment, schematically illustrated in FIG. 6, the transmitter/controller 26 may check whether the magnetic switch has been activated (block 302). If the magnetic switch 105 has been activated, a check is made to determine whether the “Op-blocker” has been activated or set to “ON” (block 306). If the “Op-blocker” is set to “ON”, the device remains inactive (step 308).

If the “Op-blocker” is set to “OFF”, the device's 10 operation may be started (block 310). A continuing check may be made to determine whether the battery voltage is above or below a predetermined level (block 312). If the battery is above the predetermined voltage level, the device's 10 operation continues. The predetermined level typically indicates depletion of the battery. Once the predetermined voltage level has been reached or exceeded, the “Op-blocker” is set to “ON” (block 314).

Though the “Op-blocker” is set to “ON” (block 214 in FIG. 5, and block 314 in FIG. 6), the device 10 may in some embodiments still continue operating (block 218 in FIG. 5, and block 318 in FIG. 6), that is until the battery dies (block 216 or 316).

Other operations and sequences of operations may be used. For example, an initiation of operation via magnetic or other switch need not be used. Conditions other than time or power supply life may be used to halt operation. Halting of operation need not be total—for example, certain components may be shut down, disabled, or partially shut down.

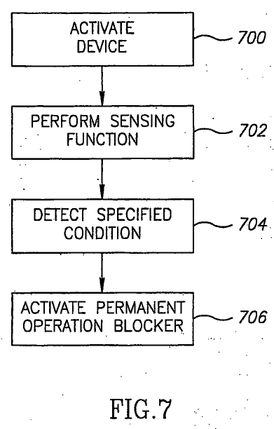

FIG. 7 is a flowchart depicting an embodiment of the present invention. In block 700, an in-vivo device 10 may be activated. For example, the device 10 may be swallowed or otherwise ingested, and for example may be activated beforehand.

In block 702, the device 10 may perform sensing functions in an in-vivo environment. For example, the device 10 may image an in-vivo lumen.

In block 704, a certain specified condition is detected. For example, a time limit may be reached, a certain number of frames may be imaged, a power level may fall to a certain level, etc.

In block 706, a disabling operation is performed such that some or all further operations of the in-vivo device 10 are blocked. For example, a memory may be burned, a memory setting may be changed, a fuse or switch may be set, a component may be disabled, etc. The disabling operation typically prevents re-activation or re-use of the device 10. In some embodiments, the device or some portion of the device may be re-activated, but may no longer be useful for its intended purpose.

Other operations or series of operations may be used.

It will be appreciated by persons skilled in the art that the present invention is not limited to what has been particularly shown and described hereinabove.

Rather the scope of the present invention is defined only by the claims which follow:

1. An in-vivo device comprising an operation blocker to prevent activation of said device after a specified condition is satisfied.

2. The device as in claim 1, wherein said operation blocker is configured to permanently prevent activation of said in vivo device after a specified condition is satisfied.

3. The device as in claim 1, wherein said operation blocker comprises a non-volatile memory configured for assuming a designated state upon said satisfaction of said specified condition.

4. The device as in claim 1, wherein said specified condition is a total elapsed time of operation of said device.

5. The device as in claim 1, wherein said specified condition is reaching a pre-defined period of operation for a current operating session of said device.

6. The device as in claim 1, wherein said specified condition is a voltage level of a power source in said device.

7. The device as in claim 1, wherein said specified condition is a receipt of a command.

8. The device as in claim 1, further comprising a timer.

9. The device as in claim 1, wherein said specified condition is satisfied by a sensor of said device detecting a pre-defined external environment.

10. The device as in claim 1, wherein said device may be activated until said specified condition is satisfied.

11. The device as in claim 1, wherein said specified condition is satisfied by a counter exceeding a predefined number of images captured by said device.

12. The device as in claim 1, wherein said operation blocker remains activated after removal or replacement of a battery.

13. The device as in claim 1, wherein said device is an autonomous in vivo device.

14. An in-vivo sensing device comprising a circuit to prevent reactivation of said device after said device has been used for a medical exam.

15. The device as in claim 14, further comprising a non-volatile memory.

16. The device as in claim 14, further comprising an operation blocker configured for preventing reactivation of said device after a specified condition has been satisfied.

17. A method for preventing reuse of an in-vivo device comprising activating a permanent operation blocker in said device upon satisfaction of a specified condition.

18. The method as In claim 17, wherein activating an operation blocker comprises burning a non-volatile memory unit into an activated position.

19. The method as in claim 17, wherein activating an operation blocker comprises melting of an insulation.

20. A method for blocking activation of an in vivo device comprising configuring a circuit to block activation of an in-vivo device upon the satisfaction of a pre-defined condition.

21. The method as in claim 20, wherein configuring a circuit comprises configuring a circuit to block activation of an in-vivo device upon a lapse of a pre-defined time period of operation of said device.

22. The method as in claim 20, wherein configuring a circuit comprises configuring a circuit to block activation of an in-vivo device upon said device capturing a pre-defined number of images.

23. The method as in claim 20, wherein configuring a circuit comprises configuring a circuit to block activation of an in-vivo device upon a voltage level in said device falling below a pre-determined voltage level.

24. The method as in claim 20, wherein configuring a circuit comprises configuring a circuit to block activation of an in-vivo device upon detection by a sensor of said device of a pre-defined external environment

25. The method as in claim 20, further comprising configuring said circuit to permit continued operation of said device after the satisfaction of a predefined condition.

26. The method as in claim 20, further comprising receiving a signal from an external command unit to activate said circuit.

27. A method of operating an autonomous in-vivo sensing device, the method comprising permanently preventing the operation of said autonomous in-vivo sensing device upon the satisfaction of a specified condition.

28. The method of claim 27, wherein the operation of said autonomous in-vivo device includes imaging.

29. The method of claim 27, wherein said preventing comprises configuring a circuit to block activation of at least a portion of the device.

30. The method of claim 27, comprising burning a memory.

31. The method of claim 27, wherein said specified condition is satisfied by a counter exceeding a predefined number of images captured by an imager.

32. The method as in claim 27, wherein said specified condition is satisfied upon the sensing of an in-vivo environmental condition.

33. The method as in claim 27, wherein said specified condition is satisfied upon a lapse of a predefined period of operation of said device.
